(19) United States
(12) Patent Application Publication
(10) Pub. No.: US 2026/0272279 A1
(43) Pub. Date: Sep. 17, 2026

(54) DENTAL MIRROR SUCTION ADAPTER

(71) Applicant: David Hazan, Maitland, FL (US)

(72) Inventor: David Hazan, Maitland, FL (US)

(21) Appl. No.: 19/544,642

(22) Filed: Feb. 19, 2026

Related U.S. Application Data
(60) Provisional application No. 63/770,726, filed on Mar. 12, 2025.

(51) Int. Cl. A61B 1/247

(52) U.S. Cl. CPC A61B 1/247

(57) ABSTRACT

A dental mirror suction adapter includes a main body having a lower body section, a middle body section and an upper body section. The lower body section includes a circular shaped element that houses a mirror. The middle body section connects the lower body section to the upper body section at an oblique angle. The upper body section includes a first opening positioned adjacent to the mirror, a hollow middle section, and a second opening located opposite to the first opening. The second opening connects to a disposable tip for a High Suction Evacuation system. A protrusion within the upper body section engages a hole in the tip to secure the HSE tip to the device. The device mirror is positionable within the mouth of a patient and any saliva, fluids and debris are sucked into the first opening, through the device and into the High Suction Evacuation system.

### CROSS-REFERENCE TO RELATED APPLICATIONS

This application claims the benefit of U.S. Application Ser. No. 63/770,726 filed on Mar. 12, 2025, the contents of which are incorporated herein by reference.

### FIELD OF THE INVENTION

The following disclosure contemplates various embodiments of a dental mirror and suction adapter device for use with high volume evacuation ports (HVE).

### BACKGROUND

The statements in this section merely provide background information related to the present disclosure and may not constitute prior art.

Dental mirrors are commonly used by dentists and oral surgeons to allow indirect vision inside the mouth of a patient. These mirrors are designed to be able to reflect light onto desired surfaces and retract soft tissue. Another tool commonly used by dentists is a Low Volume Evacuation system LVE (also referred to as a “saliva ejector) which is designed to reduce and manage the amount of saliva in the patient's mouth. Yet another tool commonly used by dentists is the High-Volume Evacuation system HVE which is used to remove large amounts of fluids and debris from the patient's mouth as would be found during procedures that involve drilling and flushing with fresh water.

The necessity of using these three separate tools requires dentists to employ the assistance of a dental assistant for virtually every procedure so that the dentist's hands are free to perform the dental work. This requirement increases the overall cost of the procedure for the patient and limits the availability of appointments to times when both the practitioner and assistant are free.

The present invention, directed to a dental mirror suction adapter differs from the conventional art in a number of aspects. The manner by which will become more apparent in the description which follows, particularly when read in conjunction with the accompanying drawings.

### SUMMARY OF THE INVENTION

The present invention is directed to a dental mirror suction adapter. One embodiment of the present invention can include a main body having a lower body section, a middle body section and a upper body section. The lower body section can include a generally circular shaped element having a mirror along the top end. The middle body section can connect the lower body section to the upper body section at an oblique angle.

In one embodiment, the upper body section can include a first opening positioned adjacent to the mirror, a hollow middle section, and a second opening located opposite to the first opening.

The second opening can include a shape and a size for receiving the end of a disposable tip for a High Suction Evacuation system. The slotted end of the tip can be inserted within the second opening, and a protrusion located within the upper body section can engage a hole in the tip.

In operation, a user can orient the device to position the mirror at any desirable angle by grasping the disposable tip. Any saliva, fluids and/or debris can be simultaneously sucked into the first opening to be removed from the patient's mouth via the High Suction Evacuation system.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of a dental mirror suction adapter device that is useful for understanding the inventive concepts disclosed herein.

FIG. 2 is a side view of the dental mirror suction adapter device in accordance with one embodiment of the invention.

FIG. 3 is a top perspective view of the dental mirror suction adapter device in accordance with one embodiment of the invention.

FIG. 4 is a perspective view of the dental mirror suction adapter device in operation, in accordance with one embodiment of the invention.

FIG. 5 is another perspective view of the dental mirror suction adapter device in operation, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described throughout this document, the term “removably secured” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

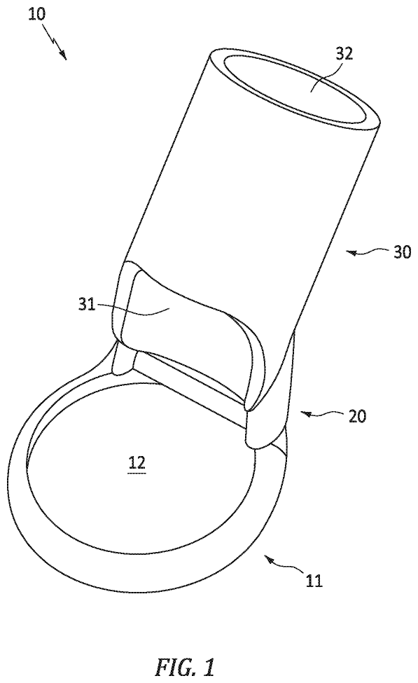

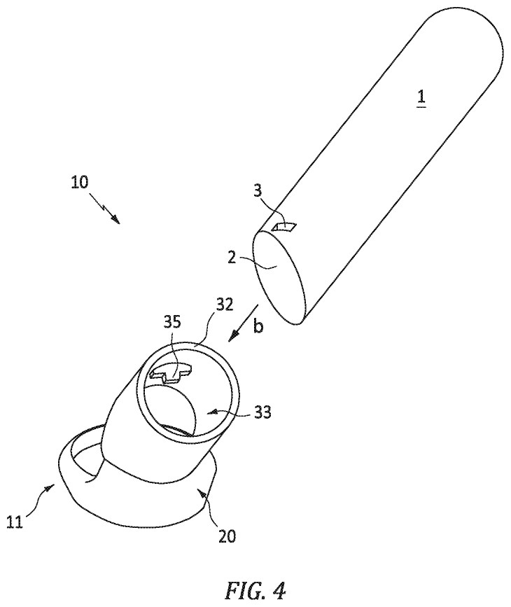

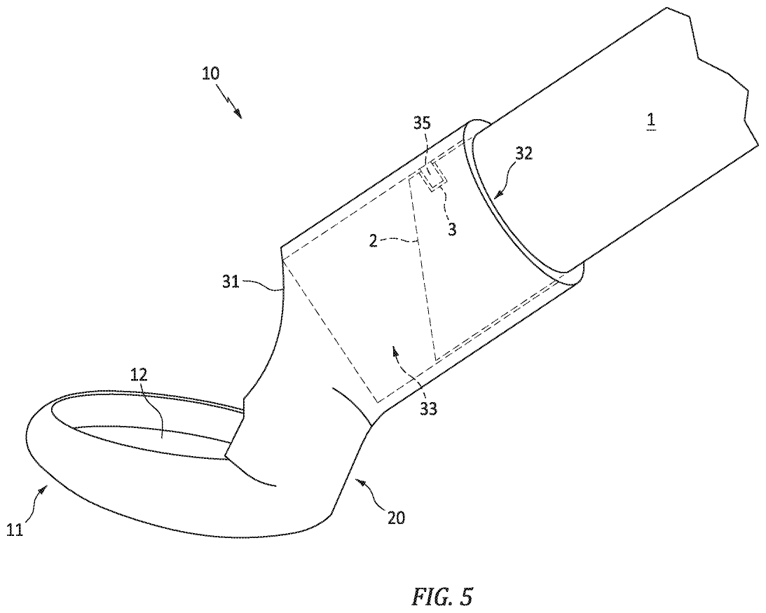

FIGS. 1-5 illustrate one embodiment of a dental mirror suction adapter 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As described below, the inventive device can be utilized by a single operator during a dental procedure to view the mouth of a patient and to remove both liquids and solids from the same using a single hand. Such a feature is advantageous, as it keeps the practitioner's other hand free to utilize additional equipment for the procedure.

As shown at FIG. 1, one embodiment, the device 10 can include an elongated curvilinear main body having a lower body section 11, a middle body section 20, and an upper body section 30.

The main body may be formed from any number of biologically inert materials that are safe for direct contact with human patients and that are relatively strong and stiff for their weight. Several nonlimiting examples include but are not limited to various metals or metal alloys (e.g., aluminum, steel, titanium, or alloys thereof), and/or various plastics or polymers (e.g., high-density polyethylene (HDPE), rigid polyvinyl chloride (PVC)), among other materials, for example.

In one embodiment, the lower body section 11 can include a generally circular shaped area having a central indentation that houses a circular mirror 12. Of course, other embodiments are contemplated wherein the mirror and lower body section include other shapes. In the preferred embodiment, the mirror can be constructed from a glycol-polyethylene terephthalate which is a clear and highly reflective thermoplastic polymer which is biocompatible and has excellent anti-fog qualities.

The mirror can be permanently affixed onto the lower section to ensure the mirror does not become separated from the device during operation and become a choking hazard to the patient. To this end, any number of different adhesives or other such materials may be utilized to connect these elements together in a manner that prevents later separation.

The middle section 20 can function to orient the lower section 11 and more precisely the reflective portion of the mirror 12 at any number of different angles relative to the upper section 30. In one embodiment, the middle section 20 can include a rigid construction that is formed integrally with the upper and lower body sections to orient the components at an oblique angle of about 45 degrees. However, other embodiments are contemplated wherein the mirror is oriented at different angles relative to the upper section.

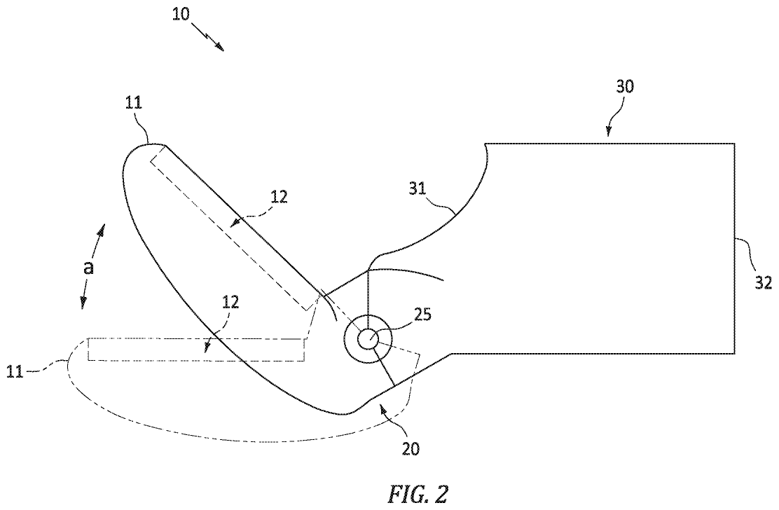

For example, FIG. 2 illustrates one embodiment of the device 10 having a hinge 25 that is positioned along the middle section 20. The inclusion of a hinge advantageously allows a user to change the overall shape of the device 10 to better fit within the confined space of a patient's mouth and to provide a user-defined angle (see arrow a) between the mirror 12 and upper section 30 when using the tool.

Although described above with regard to a single mirror positioned along the top surface of the main body, other embodiments are contemplated wherein a second mirror is located along the bottom surface of the main body such that its reflective portion is oriented directly opposite to the reflective portion of the primary mirror 12 along the top surface of the main body. Such a feature allowing a user(s) to visually examine multiple portions of a patient's mouth simultaneously, and/or to provide suction to areas of the patient's mouth other than what is directly adjacent to the mirror 12 on the top surface.

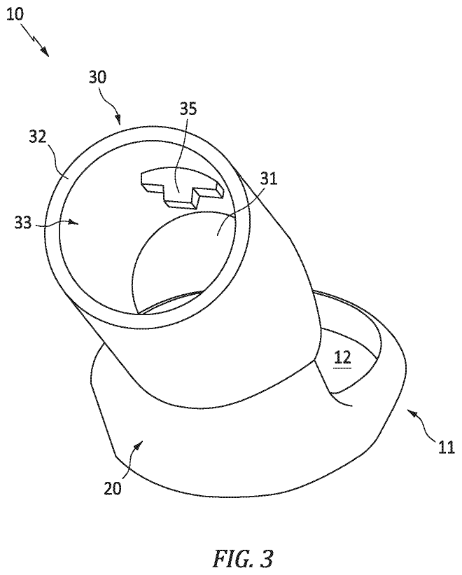

As shown best at FIGS. 3-5, the upper section 30 can include a generally tubular-shaped member having a first opening 31 along the bottom end, a second opening 32 along the top end, and a hollow interior space 33. The first opening 31 can be positioned adjacent to the mirror 12, and can function to remove both liquids (saliva and fresh water flush) and solids. Because the device is designed to remove both liquids and solids from the mirror via an HVE system, the first opening 31 will ideally include a slotted shape that extends diagonally upward relative to the mirror 12 and that includes an overall size that is between ¼ and ½ the size of the mirror itself.

The second opening 32 is designed to directly receive and engage a commercially available disposable HVE suction tip 1 having a slotted end 2 and hole 3. As shown by arrow b, the slotted portion 2 of the tip 1 can be inserted within the opening 32 until the hole 3 is aligned with and engaged by the protrusion 35 located within the second opening. When so positioned, the suction from the tip 1 will be directed through the hollow interior space 33 so as to remove debris and solids located on the mirror 12 via the first opening 31.

In engaging only the distal portion of an HVE tip the device is able to maintain a small overall dimension as the tip does not extend through the main body to engage the mirror directly. Moreover, this arrangement serves two additional important functions.

First, the protrusion securely locks the HVE tip onto the housing and prevents the device 10 from rotating independently from the tip 1. Such a feature advantageously allows a user to hold the tip 1 when positioning and orienting the device within the mouth of a patient.

Secondly, by preventing the components from rotating or otherwise moving separately from each other, the protrusion 35 maintains the slotted portion 2 of the tip in an identical orientation and alignment with the slotted shape of the first opening 31, thus ensuring maximum suction is passed from the tip 1 through the hollow main body 33 and onto the mirror 12.

In one embodiment, the device 10 (e.g., body section(s) and/or mirror) may be provided in a plurality of different sizes for use in different dental specialties as follows in Table 1:

TABLE 1

| Industry usage | Identification | Size |
|---|---|---|
| Oral surgery | #00 | 10 mm |
| Pediatric Dentistry | #3 | 20 mm |
| General dentistry | #5 | 22 mm |

Although described above with regard to particular shapes or sizes, other embodiments are contemplated having different shapes and sizes.

Accordingly, the inventive device is designed to be used singlehandedly by a practitioner, thus freeing their other hand to use other dental tools. This feature makes it possible for a dentist to work with a patient without employing a dental assistant. Moreover, when performing complex procedures such as drilling and flushing, for example, the device captures all saliva, water, and debris for evacuation by the HVE system that is attached to the device.

As to a further description of the manner and use of the present invention, the same should be apparent from the above description. Accordingly, no further discussion relating to the manner of usage and operation will be provided.

As described herein, one or more elements of the above noted device and system can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A dental device, comprising:
a main body having a lower body section, a middle body section and an upper body section,
a mirror that is positioned along the lower body section;
a first opening that is positioned along the upper body section at a location adjacent to the mirror; and
a second opening that is positioned along the upper body section at a location opposite to the first opening,
wherein the upper body section includes a hollow central portion that connects the first opening and the second opening, and
wherein the upper body section is configured to engage a suction tip.

2. The device of claim 1, wherein the middle body section includes a curvilinear shape.

3. The device of claim 2, wherein the middle body section orients the mirror at an oblique angle relative to the upper body section.

4. The device of claim 2, wherein the middle body section orients the mirror at an angle of 45 degrees relative to the upper body section.

5. The device of claim 1, wherein the main body is constructed from a biologically inert material.

6. The device of claim 1, wherein the device is configured to be used for oral surgery.

7. The device of claim 6, wherein the mirror comprises a #00 mirror having an outer diameter of about 10 mm.

8. The device of claim 1, wherein the device is configured to be used for pediatric dentistry.

9. The device of claim 8, wherein the mirror comprises a #3 mirror having an outer diameter of about 20 mm.

10. The device of claim 1, wherein the device is configured to be used for general dentistry.

11. The device of claim 10, wherein the mirror comprises a #5 mirror having an outer diameter of about 22 mm.
